(19) United States
(12) Patent Application Publication
(10) Pub. No.: US 2020/0181041 A1
(43) Pub. Date: Jun. 11, 2020

(54) CATALYTIC PROCESS FOR DIENE DIMERIZATION

(71) Applicants: TOTAL RAFFINAGE CHIMIE, Courbevoie (FR); CENTRE NATIONAL DE LA RECHERCHE SCIENTIFIQUE (CNRS), Paris (FR); UNIVERSITE CLAUDE BERNARD LYON, Villeurbanne (FR); ECOLE SUPERIEURE DE CHIMIE PHYSIQUE ELECTRONIQUE DE LYON, Villeurbanne (FR)

(72) Inventors: Mostafa TAOUFIK, Villeurbanne (FR); Kai Chung SZETO, Villeurbanne (FR); Cesar RIOS NEYRA, Villeurbanne (FR); Stéphans KRESSMAN, Communay (FR)

(73) Assignees: TOTAL RAFFINAGE CHIMIE, Courbevoie (FR); CENTRE NATIONAL DE LA RECHERCHE SCIENTIFIQUE (CNRS), Paris (FR); UNIVERSITE CLAUDE BERNARD LYON, Villeurbanne (FR); ECOLE SUPERIEURE DE CHIMIE PHYSIQUE ELECTRONIQUE DE LYON, Villeurbanne (FR)

(21) Appl. No.: 16/316,517

(22) PCT Filed: Jul. 10, 2017
(86) PCT No.: PCT/EP2017/067310

§ 371 Date: Jan. 9, 2019

(30) Foreign Application Priority Data
Jul. 12, 2016 (EP) 16305897.7

(51) Int. Cl. C07C 2/40

(52) U.S. Cl. CPC C07C 2/403; C07C 2531/02; C07C 2531/24; C07C 2523/44

(57) ABSTRACT

The disclosure relates to a process for the dimerization of conjugated diene compounds by a heterogeneous catalytic process using a supported palladium catalyst in the presence of at least one palladium activator and at least one palladium coordinating agent.

### CROSS-REFERENCE TO RELATED APPLICATIONS

This application is a National Phase Entry of International Patent Application No. PCT/EP2017/067310, filed on Jul. 10, 2017, which claims priority to European Patent Application Serial No. 16305897.7, filed on Jul. 12, 2016, both of which are incorporated by reference herein.

### TECHNICAL FIELD

The invention relates to the dimerization of conjugated diene compounds, in particular terminal conjugated diene compounds, by a heterogeneous catalytic process in a reaction medium, in order to provide dimers with a satisfying yield and/or selectivity.

### BACKGROUND

Products obtained by dimerization of conjugated dienes and further hydrogenation may be used in different fields, such as flavors and fragrances, pharmaceutical, cosmetics, solvents and lubricants applications. In cosmetic applications, hydrogenated dimers obtained from conjugated dienes may be used in creams, such as nutrient creams and medicated creams or in toilet or milky lotion, in lipstick or in face powder. In pharmaceutical applications, hydrogenated dimers obtained from conjugated dienes may be used in medical and pharmaceutical preparations such as ointments, and medical lubricating agents. As an example of a useful hydrogenated dimer, special mention can be made to squalane, isosqualane, neosqualane and crocetane.

The dimerization process of conjugated dienes is generally performed using a catalyst in the presence of a solvent. U.S. Pat. No. 4,720,576 discloses a process for dimerization of aromatic halide compounds in the presence of a platinum group metal catalyst, carbon monoxide and an alkali metal compound and/or an alkaline earth metal compound.

U.S. Pat. No. 8,669,403 discloses a process for catalytic dimerization of farnesene using a homogeneous catalytic process using complexes. This document discloses a complex of palladium. This document also discloses that heterogeneous catalysts of Pd/C, Pd/alumina or Ru/C type do not provide conversions higher than 5%. Therefore, the transposition of a homogeneous catalytic system into a heterogeneous catalyst system cannot be regarded as obvious or predictable.

In the prior art processes for the dimerization of conjugated diene compounds, a hydrogenation step is generally performed after the dimerization reaction of conjugated dienes generally in a different reactor, in particular by a hydrogenation reaction of dimers using a hydrogenation catalyst different from the dimerization catalyst, in order to obtained hydrogenated dimers. There still exists a need for the dimerization of conjugated dienes by an industrial process which would lead to dimers with a good conversion and which will be much easier to implement.

### SUMMARY

A first object of the present invention is a process for the dimerization of conjugated diene compounds comprising contacting, in a reaction medium, said conjugated diene compounds with a supported catalyst comprising at least palladium metal in the presence of at least one palladium activator and at least one palladium coordinating agent. According to an embodiment, the palladium activator is selected from protic compounds and halide compounds and mixtures thereof. Preferably, the palladium activator is selected from isopropanol, bromobenzene, iodobenzene and a combination of bromobenzene or iodobenzene with at least one of organomagnesium, organolithium, tetraalkyltin, organozinc, boronic acid, olefins such as styrene, methylacrylate, terminal alkynes.

According to an embodiment, the palladium coordinating agent is selected from phosphine and phosphite compounds. Preferably, the palladium coordinating agent is selected from triphenylphosphine, tri-ortho-tolyl phosphine, tri-meta-tolyl phosphine, tri-para-tolyl phosphine, triethylphosphine, trisisobutyl phosphine, tribenzylphosphine, dimethylphenylphosphine, biscyclohexylphenyl phosphine, bis-butylphenyl phosphine, bisphenylorthomethoxyphenyl phosphine, tris-meta-methoxy-xylyl phosphine, tris-para-methoxy-xylyl, triphenylphosphite, tris meta-methoxy-phenyl phosphine, tris ortho-methoxy-phenyl phosphine, tris para-methoxy-phenyl phosphine, bis-diphenyllphosphinoethane and bis-cyclohexylphosphinobutane.

According to an embodiment of the invention, the reaction medium comprises a phenol compound and/or a hindered phenol compound. According to an embodiment, the support of the catalyst is selected from carbon, silica, alumina, silica-alumina and zeolite, preferably carbon. According to an embodiment, the catalyst is a bimetallic catalyst PdM comprising another metal atom M different from palladium.

According to an embodiment of the invention, the process further comprises the hydrogenation of the dimers obtained after the dimerization. Preferably, the conjugated diene compounds are terminal conjugated diene compounds. According to an embodiment, the conjugated diene compounds are asymmetric conjugated diene compounds.

According to an embodiment of the invention, the conjugated diene compounds have the following formula (I):

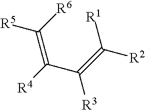

wherein R¹, R², R³, R⁴, R⁵ and R⁶ represent, independently to each other, a hydrogen atom, a halogen atom or a hydrocarbyl radical, linear, branched or cyclic, saturated or unsaturated, optionally comprising one or more heteroatoms, being understood that at least one of the Rⁱ is different from all the others Rⁱ, i being selected from 1, 2, 3, 4, 5 or 6.

According to an embodiment of the invention, the conjugated diene compounds have the following formula (II):

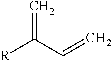

wherein R is a hydrocarbyl radical having 1 to 15 carbon atoms, preferably having 2 to 15 carbon atom, more preferably having from 5 to 15 carbon atoms, optionally substituted by one or more heteroatoms, such as nitrogen, oxygen or sulphur.

According to an embodiment of the invention, the conjugated diene compounds are selected from myrcene or farnesene. According to an embodiment of the process of the invention, the dimerization and the hydrogenation are performed within the same reactor.

An advantage of the present invention is a process that involves a supported catalyst, which is more convenient for an industrial application than homogeneous catalysts. Another advantage of the present invention is its high economical interest for an industrial process since the dimerization and the hydrogenation may be performed using the same catalyst and therefore within the same reactor. Another advantage of the present invention is its high selectivity, in particular, the process of the present invention may lead in majority to head-to-head dimers, i.e. the amount of the head-to-head dimers is higher than the amount of the other reaction products. For example, the head-to-head dimers may represent at least 40% by weight of the reaction products, preferably at least 45% by weight, more preferably at least 50% by weight of the reaction products.

To be complete, if the head-to-head dimers represent 40% by weight of the reaction products, the reaction products will not comprise one compound (different from head-to-head dimers) which alone will represent more than 40% by weight (since the process of the invention leads in majority to head-to-head dimers). An advantage of the present invention is that it may be implemented with a very small amount of solvent, leading to a more economic process. Additionally, the absence of solvent facilitates the further separation steps, improving the efficiency of the process. Further features and advantages of the invention will appear from the following description of embodiments of the invention, given as non-limiting examples, with reference to the accompanying drawing listed hereunder.

### BRIEF DESCRIPTION OF THE FIGURE

FIG. 1 represents a general formula of a conjugated diene compound.

### DETAILED DESCRIPTION

A first object of the present invention is a process for the dimerization of conjugated diene compounds comprising contacting, in a reaction medium, said conjugated diene compounds with a supported catalyst comprising at least palladium metal in the presence of at least one palladium activator and at least one palladium coordinating agent.

### Diene Compound

By “conjugated diene compounds” according to the present invention, it is to be understood a hydrocarbon compound, linear, branched or cyclic, comprising at least two conjugated carbon-carbon double bonds separated by one single bond. The hydrocarbon compound may also comprise at least one heteroatom (either in the skeleton of the main hydrocarbon chain or in side substituents or side hydrocarbon chains), such as oxygen, nitrogen or sulfur. Preferably, the hydrocarbon compound consists in hydrogen and carbon atoms. The hydrocarbon compound preferably comprises from 4 to 30 carbon atoms, more preferably from 5 to 20 carbon atoms. The hydrocarbon compound may optionally comprise one or more additional carbon-carbon double bonds, apart from the two conjugated carbon-carbon double bonds.

The conjugated diene compounds used in the present invention are preferably such that the dimerization products of said conjugated diene compounds may lead simultaneously to head-to-head dimers and head-to-tail dimers (isomers). The skilled person well knows which conjugated diene compounds can form both different isomers and which conjugated diene compounds cannot form both different isomers. In particular, the conjugated diene compounds are preferably asymmetric conjugated diene compounds, such that the dimerization reaction may lead to different dimers.

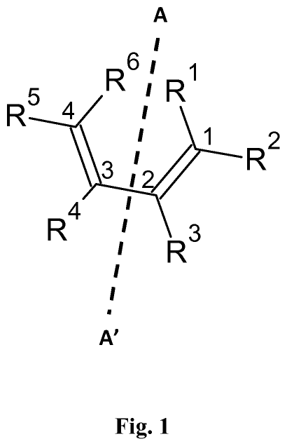

By “asymmetric conjugated diene compound”, it is to be understood a compound wherein the conjugated diene function does not comprise a plane of symmetry. The skilled person well knows what is a conjugated diene function that has a plane of symmetry or what is a conjugated diene function that has not a plane of symmetry. For example, with reference to the formula (I) below, an asymmetric conjugated diene compound is a compound which does not have a plane of symmetry between carbon atoms numbered 2 and 3, the plane of symmetry is represented by the AA′ axis in formula (I) in FIG. 1.

A conjugated diene compound used in the present invention may be represented by the following formula (I):

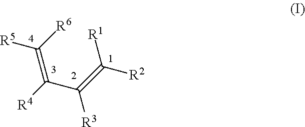

wherein R¹, R², R³, R⁴, R⁵ and R⁶ represent, independently to each other, a hydrogen atom, a halogen atom or a hydrocarbyl radical, linear, branched or cyclic, saturated or unsaturated, optionally comprising one or more heteroatoms such as oxygen, nitrogen or sulphur atoms, being understood that at least one of the Rⁱ (i being 1, 2, 3, 4, 5 or 6) is different from all the others Rⁱ, in order to obtain an asymmetric conjugated diene compounds.

Preferably, R¹, R², R³, R⁴, R⁵ and R⁶ represent, independently to each other, a hydrogen atom or a hydrocarbyl radical having from 1 to 20 carbon atoms, preferably without heteroatoms, being understood that at least one of the Rⁱ (i being 1, 2, 3, 4, 5 or 6) is different from all the others Rⁱ. According to an embodiment, R¹, R², R³ and R⁴ are hydrogen atoms; R⁵ is different from R⁶; and R⁵ and R⁶ are selected from a hydrogen atom or a hydrocarbyl radical having from 1 to 20 carbon atoms, optionally comprising heteroatom(s). In the above formula (I) also represented in FIG. 1, the four carbon atoms of the conjugated diene function have been numbered from 1 to 4.

A “head-to-head dimer” is well known for the skilled person. For example, with reference to the formula (I) above, a head-to-head dimer is a dimer obtained by reaction between a 1-2 carbon-carbon double bond of one conjugated diene compound and the 1-2 carbon-carbon of another conjugated diene compound. A “head-to-tail dimer” is well known for the skilled person. For example, with reference to formula (I) above, a head-to-tail dimer is a dimer obtained by reaction between a 1-2 carbon-carbon double bond of one conjugated diene compound and the 3-4 carbon-carbon double bond of another conjugated diene compound. According to an embodiment, the conjugated diene compounds are terminal conjugated diene compounds.

According to an embodiment, the terminal conjugated diene compounds have the following formula (II):

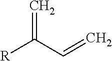

wherein R is a hydrocarbyl radical, linear, branched or cyclic, saturated or unsaturated having 1 to 20 carbon atoms, optionally comprising one or more heteroatoms (either in the skeleton of the main hydrocarbon chain or in side substituents or side hydrocarbon chains), such as nitrogen, oxygen or sulphur. Preferably, R is a hydrocarbyl radical having from 2 to 18 carbon atoms, more preferably having from 4 to 15 carbon atoms, even more preferably having from 6 to 12 carbon atoms.

According to an embodiment, the conjugated diene compounds are chosen from terpenes, such as myrcene or beta-farnesene, beta-phellandrene or alpha-terpinene, preferably from myrcene, beta-farnesene or beta-phellandrene, more preferably from myrcene or beta-farnesene. Myrcene refers to a compound having the following formula (III):

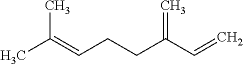

Beta-farnesene refers to a compound having the following formula (IV):

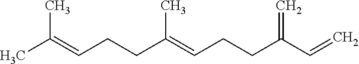

Terpenes are molecules of natural origin, produced by numerous plants, in particular conifers. By definition, terpenes (also known as isoprenoids) are a class of hydrocarbons bearing as the base unit an isoprene moiety (i.e. 2-methyl-buta-1,3-diene). Isoprene [CH₂═C(CH₃)CH═CH₂] is represented below (V):

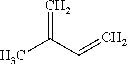

Terpenes may be classified according to the number n (integer) of isoprene units of which it is composed, for example:
n=2: monoterpenes (C₁₀), such as myrcene;
n=3: sesquiterpenes (C₁₅), such as farnesene;
n=4: diterpenes (C₂₀).

Alpha-terpinene is a cyclic terpene having two conjugated carbon-carbon double bonds and refers to a compound having the following formula (VI):

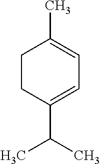

According to an embodiment, the dimerization reaction is performed with conjugated dienes of same chemical nature. According to another embodiment, the dimerization reaction is performed with conjugated dienes of different chemical natures. Preferably, the dimerization reaction is performed with conjugated dienes of same chemical nature.

The process according to the present invention is performed in a reaction medium, said reaction medium may comprise a hydrocarbon solvent or may be free of hydrocarbon solvents. According to the present invention, by “hydrocarbon solvent”, it is to be understood an additional component, different from the conjugated diene compounds, different from the catalyst(s), different from the palladium activator and different from the palladium coordinating agent. It is to be understood that the palladium activator for example isopropanol may also play the role of a solvent.

### Catalyst

The catalyst used in the dimerization process of the present invention is a supported catalyst comprising palladium atoms. The support of the catalyst may be carbon, alumina, silica, silica-alumina or zeolite, preferably carbon. According to an embodiment, the supported catalyst is selected from palladium on carbon (Pd/C) catalyst, palladium on alumina catalyst, palladium on zeolite catalyst, palladium on silica-alumina catalyst, preferably from palladium on carbon (Pd/C) catalysts.

According to an embodiment of the invention, bimetallic catalysts are used of the type PdM, wherein M is a second metal different from palladium. The second metal M may be selected from copper, gold or silver. Bimetallic catalysts may provide a catalyst having an improved activity.

Supported palladium-based catalysts are commercially available. In particular Pd/C catalysts are commercially available and are generally in the form of a mixture of Pd(II) and Pd(0), being understood that generally the surface of the catalyst is oxidized. According to a well-known process for the skilled person, the oxidation degree of the metal may be reduced to zero by the action of hydrogen.

### Reaction Medium

The reaction medium wherein the dimerization reaction takes place comprises the supported palladium catalyst, the conjugated dienes, at least one palladium activator and at least one palladium coordinating agent. By “palladium activator”, it is to be understood a compound able to extract at least a part of the palladium from its support and reduce the Pd. By “palladium coordinating agent”, it is to be understood a compound able to give at least one electron to the palladium. The palladium coordinating agent can be a ligand of the palladium, and it can be of the monodentate or of the polydentate type, in particular of the bidentate type.

The combined function of the activator and the coordinating agent is to provide a partial solubilisation/dissolution, into the reaction medium, of the palladium metal initially present in or on the support. Preferably, the reaction medium comprises at least 50% by weight of palladium activator, preferably at least 70% by weight, more preferably at least 90% by weight, still more preferably at least 99% by weight of palladium activator, based on the total weight of the reaction medium.

According to an embodiment, the palladium activator is selected from a protic compound, such as primary or secondary alcohols, thiol (R′SH, wherein R′ is a hydrocarbyl radical) or amines, and from halide compounds, such as aryl halide compounds, alone or in a mixture with organomagnesium (for example of type QMgX wherein X is a halogen atom and Q is an organic radical having from 1 to 42 carbon atoms), organolithium (for example of type Q′LiX′ wherein X′ is a halogen atom and Q′ is an organic radical having from 1 to 42 carbon atoms), tetraalkyltin (wherein the alkyl may have from 1 to 42 carbon atoms), boronic acid, methyl acrylate, olefins (such as styrene), terminal alkyne (wherein the carbon-carbon triple bond is in terminal position of the hydrocarbon chain and wherein the alkyne may comprises from 2 to 42 carbon atoms).

In some embodiments, the palladium activator may also play the role of a solvent during the reaction, in particular, when it is added in a high amount. By “protic compound”, it is to be understood a compound that has a labile H⁺. Preferably, the palladium activator is selected from isopropanol, bromobenzene and iodobenzene, more preferably the palladium activator is isopropanol.

According to an embodiment, the palladium coordinating agent is selected from phosphine or phosphite compounds. Within the meaning of the present invention, by “phosphite compound” it is to be understood a phosphite molecule of formula PO³⁻ and a phosphite derivative such as phosphite of formula P(OL⁴)₃ wherein L⁴ represent independently to each other an organic radical, preferably a radical selected from linear or branched alkyls having from 1 to 12 carbon atoms, linear or branched alkenyls having from 2 to 12 carbon atoms, or aryl optionally substituted having from 6 to 15 carbon atoms. Within the meaning of the present invention, by “phosphine compound” it is to be understood a phosphine molecule of formula PH₃ and a phosphine derivative such as an organophosphorus ligand of formula PL¹L²L³ wherein L¹, L², L³ represent independently to each other an organic radical. According to an embodiment of the invention, the phosphine compound may be selected from compounds having the formula PL¹L²L³, wherein L¹, L², L³ represent independently to each other a hydrogen atom, a halogen atom, a radical selected from linear or branched alkyls, linear or branched alkenyls or aryl optionally substituted, preferably, L¹, L², L³ represent independently to each other a hydrogen atom, a halogen atom, a radical selected from linear or branched alkyls having from 1 to 12 carbon atoms, linear or branched alkenyls having from 2 to 12 carbon atoms, or aryl optionally substituted having from 6 to 15 carbon atoms.

According an embodiment, the phosphine compounds are selected from triphenylphosphine [PPh₃], tri-ortho-tolyl phosphine [(o-tolyl)₃P], tri-meta-tolyl phosphine [(m-tolyl)₃P], tri-para-tolyl phosphine [(p-tolyl)₃P], triethylphosphine [PEt₃], Trisisobutyl phosphine [tBu₃P], tribenzylphosphine [PBn₃], dimethylphenylphosphine [PMe₂Ph], biscyclohexylphenyl phosphine [PhPCy₂], bis-butylphenyl phosphine [PhPBu₂], bisphenylorthomethoxyphenyl phosphine [(o-MeOPh)PPh₂], tris-meta-methoxy-xylyl phosphine, [m-MeO-xyl)₃P], tris-para-methoxy-xylyl [p-MeO-xyl)₃P], triphenylphosphite [P(OPh)₃], tris meta-methoxy-phenyl phosphine [(m-MeOPh)₃P], tris ortho-methoxy-phenyl phosphine [o-MeOPh)₃P], tris para-methoxy-phenyl phosphine [p-MeOPh)₃P], bis-diphenyllphosphinoethane [dppe], bis-cyclohexylphosphinobutane [dcpb], preferably from triphenylphosphine.

According to the invention, other coordinating agents, different from the phosphine and phosphite compounds described above, may be used, alone or in combination. Examples of such other coordinating agents are:

(i) Monodentate pyridine type ligands, such as:

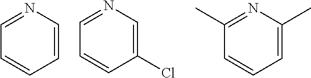

(ii) Bidentate ligands such as phosphine-phosphines, phosphines-amine, phosphines-pyridine, and phosphine-sulfurs
(iii) Bidentate bis-phosphines or bis-phosphite ligands, such as:

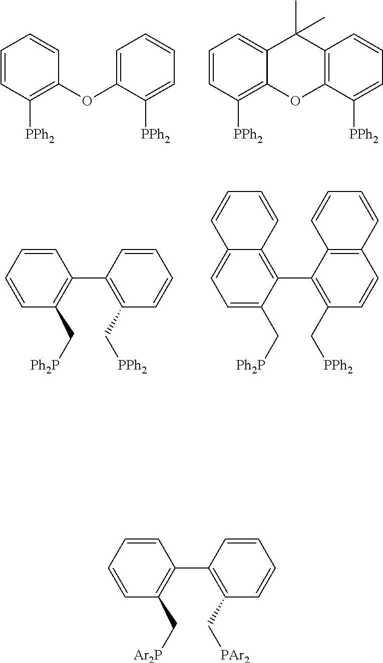

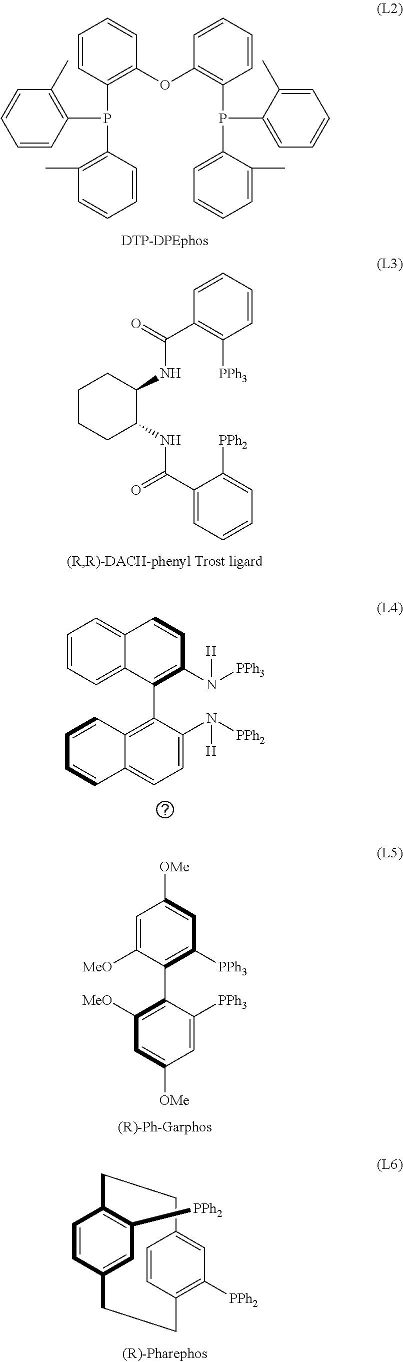

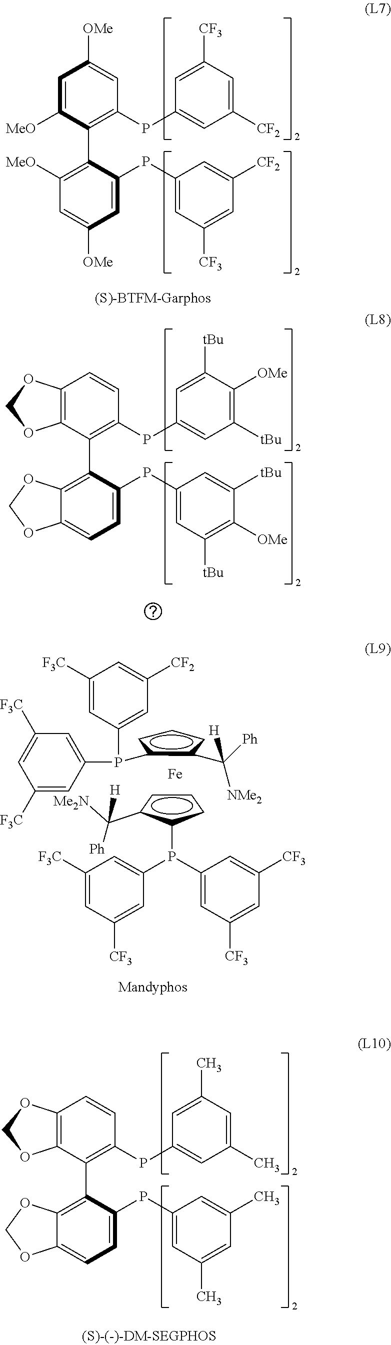

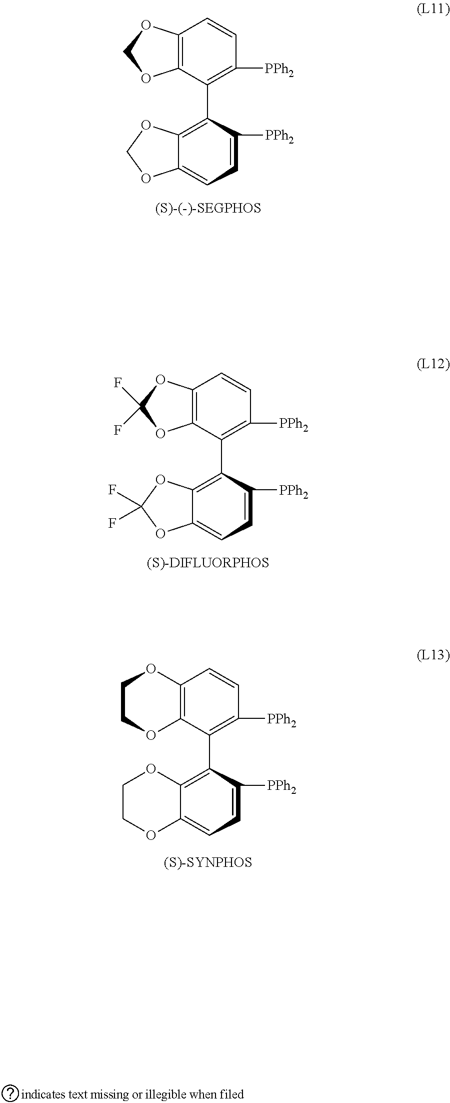

(iv) Phosphine-pyridine ligands, such as:

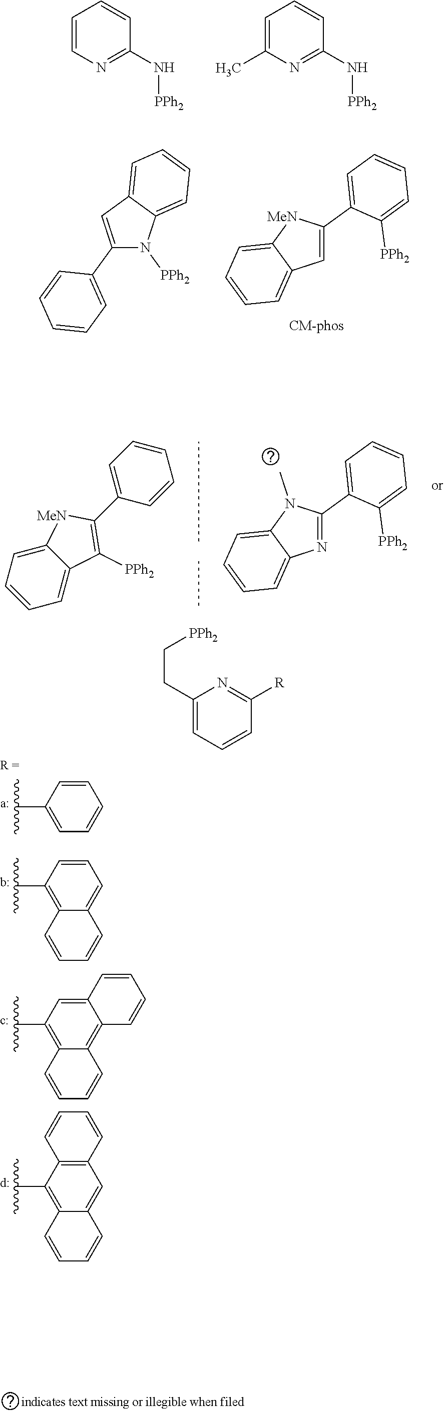

(v) N-Heterocyclic Carbene (NHC) ligands
Said NHC ligand may be synthetized as per several methods, which are:
1—deprotonation of imidazolium salts with a strong base (equation 1 below)
2—reduction of the thione with potassium (equation 2 below)
3—thermal decomposition of the alcohol, CO₂ or methylene chloride or pentafluorobenzene (equations 3 et 4 below)

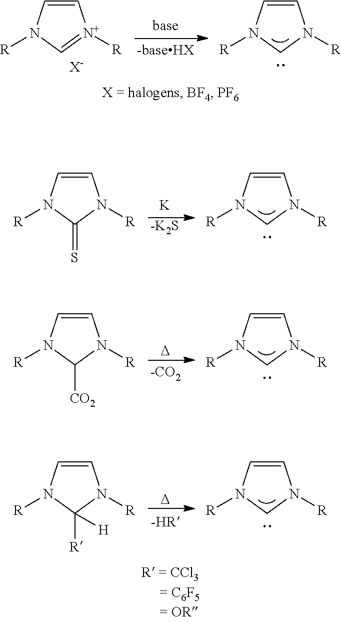

Examples of NHC monodentate ligand are

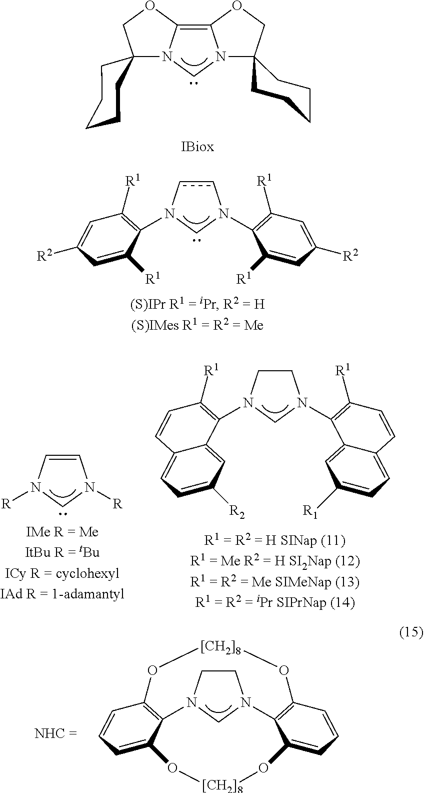

Examples of bidentate NHC-phosphine ligands are

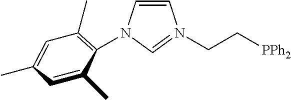

Examples of bidentate NHC-NHC ligands are

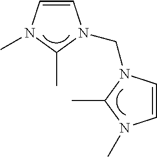

(vi) other monodentate NHC and imine or pyridine ligands:

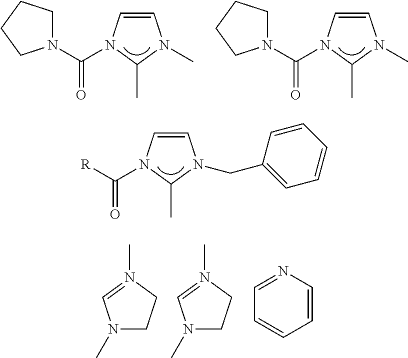

According to an embodiment of the process of the invention, the molar ratio between the palladium coordinating agent and the palladium ranges from 0.5 to 3, preferably from 0.75 to 2.75, more preferably from 1 to 2.5, even more preferably from 1.5 to 2.0. According to a preferred embodiment, the palladium coordinating agent is selected from phosphine compounds. According to a specific embodiment of the invention, the process is performed in a reaction medium comprising a primary or a secondary alcohol, such as isopropanol, as a palladium activator and a phosphine compound, such as triphenylphosphine, as a palladium coordinating agent.

According to an embodiment, the dimerization process according to the invention comprises the reaction between at least two conjugated diene compounds in a reaction medium comprising a hydrocarbon solvent. Within the meaning of the present invention, “hydrocarbon solvents” refers to non protic compounds. They are solvents for the diene compounds. According to this embodiment of the invention, the selected hydrocarbon for the solvent of the reaction medium is different from the diene compounds described above and preferably different from the palladium activator agent.

The hydrocarbon solvents comprised in the reaction medium may be chosen from a linear, a branched or a cyclic hydrocarbon. For example, the hydrocarbon solvents may be chosen from pentane, heptane, hexane, cyclohexane, toluene and o-xylene. According to another embodiment, the dimerization process according to the invention comprises the reaction between at least two conjugated diene compounds in a reaction medium free of hydrocarbon solvents.

### Other Optional Additives

The process of dimerization according to the present invention may be performed in a reaction medium comprising one or more other additives, different from the conjugated diene compounds, different from the catalyst(s), different from the palladium activator(s) and different from the palladium coordinating agent(s). According to an embodiment, the reaction medium, wherein the dimerization reaction takes place, further comprises at least one additive selected from phenol and hindered phenol compounds. Within the meaning of the present invention, the phenol and hindered phenol compounds are not considered as a “solvent” as defined above. By “hindered phenol compound” according to the present invention, it is to be understood a phenol substituted with one or more substituents.

According to an embodiment of the invention, the hindered phenol compounds are selected from compounds responding to the following formula (VII):

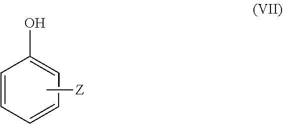

wherein Z represents one or more substituents, independently to each other, selected from hydrocarbyl radicals, linear branched or cyclic, optionally comprising one or more heteroatoms, such as sulphur, nitrogen or oxygen.

Preferably, the hindered phenol compounds of formula (VII) are mono- or di-substituted by one or two Z substituents, preferably selected from alkyl radical having from 1 to 15 carbon atoms, and said alkyl radical being linear, branched or cyclic. The substituents of the hindered phenol compound may be chosen from methyl, ethyl, propyl, isopropyl, phenyl, tertiobutyl or mesityl groups, for example from methyl, ethyl or propyl groups. Preferably, the hindered phenol compound is substituted in ortho position of the OH function of the phenol by one or two substituents.

According to an embodiment of the invention, the reaction medium comprises an additive selected from phenol, dimethylphenol, mesitylphenol or 2,6-di-tert-butyl-4-methylphenol. Preferably, the additive is a phenol, i.e. a non-substituted phenol. According to an embodiment of the invention, the pKa of the phenol based additive is preferably higher than or equal to 9.9. The inventors surprisingly found that the addition of a phenol compound in the reaction medium improved the yield of the dimerization reaction. According to an embodiment, the phenol compound represents, by weight, from 0.2 to 2%, preferably from 0.4 to 1%, ideally around 0.6%, of the reaction medium (solvent if any+diene+phenol+activator+coordinating agent). Alternatively, the phenol based additive/diene compound weight ratio at the beginning of the reaction may range from 0.2 to 9.0, preferably from 1.0 to 6.0.

According to an embodiment of the invention, a base which can be organic or inorganic is added into the reaction medium, preferably at the end of the dimerization reaction. Said base may be selected from triethylamine, sodium carbonate, potassium carbonate, sodium acetate and sodium formiate. The base may help to increase the Pd concentration in solution during the activation and also for the re-deposition of the palladium metal in or on the support, after its partial dissolution/extraction.

### Reaction Process

The reaction of dimerization is preferably performed at a temperature ranging from 25° C. to 150° C., preferably from 25° C. to 140° C., preferably from 50° C. to 120° C. At higher temperatures, there is a risk that the diene polymerizes. The reaction of dimerization is preferably performed in an inert gas atmosphere, for example in argon or nitrogen atmosphere, preferably at atmospheric pressure. The reaction of dimerization is preferably performed during at least 5 hours, preferably at least 8 hours, more preferably during from 8 to 36 hours, ideally from 12 to 24 hours.

The reaction of dimerization is preferably performed with a molar ratio conjugated dienes/catalyst ranging from 200 to 30000, preferably from 500 to 25000, more preferably from 1000 to 20000, even more preferably from 2000 to 10000. The reaction of dimerization is preferably performed with a molar ratio phenol based additive/catalyst ranging from 10 to 3200, preferably from 20 to 1500, more preferably from 60 to 640. The process can be a batch process, a semi-batch process or a continuous process and preferably takes place in a stirred reactor. Upon completion of the reaction, the resulting dimerization product can be separated off from the reactor stream in a manner known per se, for instance by distillation, absorption, etc.

The dimerization product can further be submitted to a hydrogenation reaction using the same catalyst as the catalyst used for the dimerization reaction. Preferably, for the hydrogenation reaction, the palladium catalyst, such as Pd/C, is in a reduced form, i.e. the palladium atom has a zero oxidation degree. A stream of hydrogen may be added in order to reduce the palladium catalyst, such as Pd/C, and favor the hydrogenation reaction. Upon completion of the reaction, the resulting hydrogenation products can be separated off from the reactor stream in a manner known per se, for instance by distillation, absorption, etc.

According to an embodiment of the process, the process comprises the following successive steps:
a) providing a reaction medium comprising the supported palladium catalyst, at least a part of the palladium activator and at least a part of the palladium coordinating agent; said reaction medium being substantially free of conjugated diene compounds,
b) introducing the conjugated diene compounds into the reaction medium formed in step a) in order to perform the reaction of dimerization,
c) optionally hydrogenating the dimers obtained at the end of step b),
d) recovering the, optionally hydrogenated, dimers.

According to a preferred embodiment, the dimerization reaction and the hydrogenation reaction take place in only one reactor. The process of the invention has the advantage of performing the dimerization reaction and the hydrogenation reaction with the same catalyst, and therefore they can be performed in the same reactor. Indeed, the inventors surprisingly found that the supported palladium-based catalyst, such as Pd/C, can be used for performing the dimerization of conjugated diene compounds, in particular of terminal conjugated diene compounds that optionally contain at least one additional carbon-carbon double bond.

According to another embodiment, the dimerization reaction and the hydrogenation reaction are performed in two dynamic reactors in series. After hydrogenation, hydrogenated dimers are obtained, such as squalane or isosqualane, crocetane, hydrogenated dimer of alpha-terpinene, hydrogenated dimer of beta-phellandrene. Preferably, dimers obtained after the hydrogenation are saturated dimers.

The process of the invention leads to reaction products containing the desired dimers which are mainly composed of head-to-head dimers. However, a dimerization reaction of conjugated diene compounds may lead to different reaction products. The reaction products may be dimers, trimers, etc. . . . Different dimers may be obtained, such as head-to-head dimers or head-to-tail dimers (isomers) or also cyclic dimers from cyclization reaction (Diels-Alder reaction).

The “selectivity for compound X” refers to the amount of compound X formed in the dimerization reaction based on the total amount of products formed. The selectivity is expressed as a percentage by weight. Preferably, the head-to-head dimer obtained represents at least 40% by weight of the reaction products, preferably at least 45% by weight of the reaction products, more preferably at least 50% by weight of the reaction products. In particular, the head-to-head dimers are generally present in greater proportions than the other reaction products. Within the meaning of the present invention, the expression “reaction products” refers to all the products obtained at the end of the reaction (dimers, trimers, etc). The conjugated diene compounds (the reactants of the reaction) are not taken into account when we deal with the reaction products.

### EXAMPLES

### Comparative Example C1: Dimerization of Farnesene Using a Pd/C Catalyst Without Phosphine

β-Farnesene was degassed via four freeze-pump-thaw cycles and used without further purification for the dimerization reaction with Pd/C catalyst. Pd/C catalyst (200 mg, catalyst used as received without any pretreatment), farnesene (molar ratio farnesene/Pd=3000, 115 g) were charged in a 100 mL schlenk. Then, 12 mL of solvent (isopropanol) was added under atmospheric pressure of Argon or nitrogen to this mixture and stirred for 12 h at isopropanol reflux (boiling point 82.6° C.). No phenol is present in the reaction medium. Finally, the crude of the dimerization reaction was filtrated through a silica path on Büchner fritted disc funnel and washed several times with toluene. The solvent was evaporated in the rotavapor.

The crude of the dimerization reaction (0.089 g) was charged in a stainless steel autoclave with 10 wt % Pd/C (150 mg), 5 mL of toluene, 40 bar of H₂ and stirred for 12 h at 85° C. After that, an internal standard nonadecane (80 mg) was added to the hydrogenated mixture and an aliquot was injected in the GC-FID to obtain the conversion and selectivity on squalane and isosqualane. The conversion was mainly calculated based on the latter method unless further specification. The conversion has been mentioned in table 1 below. Given the very low conversion, the selectivity has not been evaluated.

### Examples 1 to 3: Dimerization of Farnesene Using a Pd/C Catalyst in the Presence of Phosphine

β-Farnesene was degassed via four freeze-pump-thaw cycles and used without further purification for the dimerization reaction with Pd/C catalyst. Pd/C catalyst (200 mg, catalyst used as received without any pretreatment), PPh₃ phosphine compound (50 mg), farnesene (molar ratio farnesene/Pd=3000, 115 g) were charged in a 100 mL schlenk. Then, 12 mL of solvent (isopropanol) was added under atmospheric pressure of Argon or nitrogen to this mixture and stirred for 12 h at isopropanol reflux (boiling point 82.6° C.). No phenol is present in the reaction medium. Finally, the crude of the dimerization reaction 10 was filtrated through a silica path on Büchner fritted disc funnel and washed several times with toluene. The solvent was evaporated in the rotavapor.

The crude of the dimerization reaction (0.089 g) was charged in a stainless steel autoclave with 10 wt % Pd/C (150 mg), 5 mL of toluene, 40 bar of H2 and stirred for 12 h at 85° C. After that, an internal standard nonadecane (80 mg) was added to the hydrogenated mixture and an aliquot was injected in the GC-FID to obtain the conversion and selectivity on squalane and isosqualane. The conversion was mainly calculated based on the latter method unless further specification.

For example 1, the reaction medium comprises 12 mL of isopropanol as activator and solvent and does not comprise phenol. For example 2, the reaction medium comprises 12 mL of isopropanol as activator and solvent and 0.3 mL of phenol (i.e. Phenol/Pd molar ratio of 200). For example 3, the reaction medium comprises 12 mL of isopropanol as activator and solvent and 1 mL of phenol (i.e Phenol/Pd molar ratio of 630).

The head-to-head dimer obtained after hydrogenation of farnesene is the squalane which can be represented by the following formula:

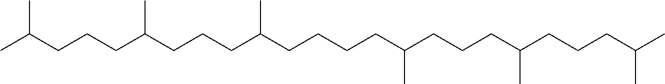 [SMILES: CC(C)CCCC(C)CCCC(C)CCCCC(C)CCCC(C)CCCC(C)C]

The conversion and selectivity are indicated in the table 1 below.

TABLE 1
conversion and selectivities

| | | Selectivity | | | | |
|---|---|---|---|---|---|---|
| | | | Iso- | | cyclic | Molar ratio |
| | Conversion | Squalane | squalane | Trimer | dimers | squalane/ |
| | (%) | (%) | (%) | (%) | (%) | isosqualane |
| C1 | 7 | n.m. | n.m. | n.m. | n.m. | n.m. |
| 1 | 55 | 87.1 | 6.3 | 0.5 | 6.1 | 13.8 |
| 2 | 57 | 73.7 | 19.3 | 0.4 | 6.6 | 3.8 |
| 3 | 95 | 54.1 | 39.2 | 3.8 | 2.9 | 1.4 |

n.m. = not measured

As illustrated in the above table 1, it can be seen that the dimerization and hydrogenation of farnesene can be performed using a Pd/C catalyst in the presence of phosphine compounds with good conversion, in particular with a conversion higher than 50% and which can be as high as 95% (see example 3). By comparing examples 2 and 3, we can see that the addition of a phenol compound allows further increasing the conversion of farnesene. The process of the invention has the great advantage of allowing the dimerization reaction and the hydrogenation reaction to be performed with the same catalyst which facilitates the industrial implementation and reduces the costs of the process.

### Example 4

This example aims at evidencing the ability of both isopropanol and bromobenzene to extract palladium from a Pd catalyst as per the invention.

### Isopropanol:

In the glovebox, about 200 mg of Pd/C (10 wt % Pd, Aldrich) in a solution containing PPh3 (49.98 mg), phenol (1.0744 g) and farnesene (4.58 g). Then, 10 ml of dried and oxygen-free isopropanol was added to the solution and the schlenk was immediately connected to a reflux column. The solution was stirred and heated with and oil bath set at 115° C. (external temperature). After 2 hours, a sample of the solution was filtered and the filtrate was submitted to elemental analysis for Pd determination. Pd concentration was found to be 447 mg/kg, that corresponds to 0.057 mmol of Pd has been extracted from Pd/C.

### Bromobenzene:

In the glovebox, about 200 mg of Pd/C (10 wt % Pd, Aldrich) in a solution containing PPh3 (50.52 mg), phenol (1.0699 g), bromobenzene (1.68 μl, 0.016 mmol), styrene (1.83 μl, 0.016 mmol), tri-octylamine (14.9 μl, 0.032 mmol) and farnesene (4.58 g). Then, 10 ml of dried and oxygen-free heptane was added to the solution and the schlenk was connected to a reflux column. The solution was stirred and heated with and oil bath set at 115° C. (external temperature). After 2 hours, a sample of the solution was filtered and the filtrate was submitted to elemental analysis for Pd determination. Pd concentration was found to be 243 mg/kg, that corresponds to 0.018 mmol of Pd (expected: 0.016 mmol) has been extracted from Pd/C.

1. A process for dimerization of conjugated diene compounds comprising contacting, in a reaction medium, the conjugated diene compounds with a supported catalyst comprising at least palladium metal in the presence of at least one palladium activator and at least one palladium coordinating agent.

2. The process according to claim 1, wherein the palladium activator is selected from protic compounds and halide compounds and mixtures thereof.

3. The process according to claim 2, wherein the palladium activator is selected from isopropanol, bromobenzene, iodobenzene and a combination of bromobenzene or iodobenzene with at least one of organomagnesium, organolithium, tetraalkyltin, organozinc, boronic acid, or olefins.

4. The process according to claim 1, wherein the palladium coordinating agent is selected from phosphine and phosphite compounds.

5. The process according to claim 4, wherein the palladium coordinating agent is selected from triphenylphosphine, tri-ortho-tolyl phosphine, tri-meta-tolyl phosphine, tri-para-tolyl phosphine, triethylphosphine, trisisobutyl phosphine, tribenzylphosphine, dimethylphenylphosphine, biscyclohexylphenyl phosphine, bis-butylphenyl phosphine, bisphenylorthomethoxyphenyl phosphine, tris-meta-methoxy-xylyl phosphine, tris-para-methoxy-xylyl, triphenylphosphite, tris meta-methoxy-phenyl phosphine, tris orthomethoxy-phenyl phosphine, tris para-methoxy-phenyl phosphine, bis-diphenyilphosphinoethane and bis-cyclohexylphosphinobutane.

6. The process according to claim 1, wherein the reaction medium comprises a phenol compound and/or a hindered phenol compound.

7. The process according to claim 1, wherein the support of the catalyst is selected from carbon, silica, alumina, silica-alumina and zeolite.

8. The process according to claim 1, wherein the catalyst is a bimetallic catalyst PdM comprising another metal atom M different from palladium.

9. The process according to claim 1, further comprising hydrogenation of dimers obtained after the dimerization.

10. The process according to claim 1, wherein the conjugated diene compounds are terminal conjugated diene compounds.

11. The process according to claim 1, wherein the conjugated diene compounds are asymmetric conjugated diene compounds.

12. The process according to claim 1, wherein the conjugated diene compounds have the following formula (I): 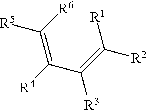
wherein R¹, R², R³, R⁴, R⁵ and R⁶ represent, independently to each other, a hydrogen atom, a halogen atom or a hydrocarbyl radical, linear, branched or cyclic, saturated or unsaturated, optionally comprising one or more heteroatoms, being understood that at least one of the Rⁱ is different from all the others Rⁱ, i being selected from 1, 2, 3, 4, 5 or 6.

13. The process according to claim 1, wherein the conjugated diene compounds have the following formula (II): 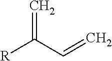
wherein R is a hydrocarbyl radical having 1 to 15 carbon atoms, optionally substituted by one or more heteroatoms.

14. The process according to claim 1, wherein the conjugated diene compounds are selected from myrcene or farnesene.

15. The process according to claim 9, wherein the dimerization and the hydrogenation are performed within the same reactor.
